(19) United States
(12) Patent Application Publication
(10) Pub. No.: US 2026/0048071 A1
(43) Pub. Date: Feb. 19, 2026

(54) 12-LIPOXYGENASE INHIBITORS FOR THE TREATMENT OF LUPUS

(71) Applicant: VERALOX THERAPEUTICS INC., Frederick, MD (US)

(72) Inventors: David MALONEY, Point of Rocks, MD (US); Matthew BOXER, New Market, MD (US)

(21) Appl. No.: 19/128,392

(22) PCT Filed: Nov. 10, 2023
(86) PCT No.: PCT/US2023/079411

§ 371 Date: May 8, 2025

Related U.S. Application Data
(60) Provisional application No. 63/383,287, filed on Nov. 11, 2022.

(51) Int. Cl. A61K 31/63; A61K 31/635

(52) U.S. Cl. CPC A61K 31/63; A61K 31/635

(57) ABSTRACT

Disclosed is a method of treating lupus using therapeutically effective amount of a selective 12-lipoxygenase inhibitor. In some embodiments, the 12-lipoxygenase inhibitor is N-(benzo[d]thiazol-2-yl)-4-[(2-hydroxy-3-methoxybenzyl)amino]benzenesulfonamide or a related benzylsulfonamide compound.

### BACKGROUND OF THE INVENTION

Lupus is a chronic autoimmune disease that causes systemic inflammation in multiple organs. The etiology of lupus is unknown, but it is suspected to arise from multiple causes including genetics, biological sex (women are more afflicted than men), and environmental factors. Typically, lupus is characterized by active flare ups followed by periods of remission. Lupus affects skin and joints, but also can also affect other organs including kidneys, pleura, pericardium, and brain. Skin problems include rashes, hair loss and mouth sores and these issues can be exacerbated by exposure to sunlight. Joint problems include swelling and stiffness of joints and arthritis. Kidney problems associated with lupus include lupus nephritis and disorders of the urinary system including proteinuria. Lupus less frequently affects the brain although headaches, dizziness, and seizures are reported in lupus patients with brain involvement. Red blood cell count is frequently lower in lupus patients, too. Inflammation of the heart and lung pleura and pericardium leads to pleurisy and pericarditis in lupus patients. People with lupus generally feel ill and fatigued and suffer weight loss.

Types of lupus include, for example, systemic lupus erythematosus (SLE), cutaneous lupus erythematosus (CLE) (CLE includes, e.g., acute cutaneous lupus erythematosus (ACLE), subacute cutaneous lupus erythematosus (SCLE), intermittent cutaneous lupus erythematosus, and chronic cutaneous lupus), lupus nephritis, drug-induced lupus, and neonatal lupus. About 70% of all cases of lupus are SLE. In CLE patients, symptoms are generally limited to the skin. More than 90% of people with lupus are women.

Lupus is typical treated with NSAIDS, steroid, immunosuppressants, and hydroxychloroquine, along with lifestyle modifications such as diet and sun protection to avoid flare ups. There is an evolving need for effective treatments.

Lipoxygenases are a class of non-heme iron-containing enzymes which regio- and stereospecifically oxidize polyunsaturated fatty acid substrates such as arachidonic acid (AA) and linoleic acid (LA). (Solomon, et al. Chem. Biol. 1997.4, 795-808; Brash, J. Biol. Chem. 1999, 274, 23679-23682.) The position at which these cis, cis-1,4-pentadiene substrates are oxidized correspond to the requisite lipoxygenase, with the three major human lipoxygenases: 5-LOX, 12-LOX, and 15-LOX-1, oxidizing the C-5, C-12 and C-15 positions respectively. Lipoxygenases are involved in the first committed step in a cascade of metabolic pathways and the products of these enzymes (eicosanoids) are precursors of hormones such as leukotrienes and lipoxins, which mediate a wide array of cellular functions (Serhan, et al. Chem. Rev. 2011, 111, 5922-5943). Consequently, the lipoxygenase enzymes and their bioactive metabolites (e.g. hydroxyeicosatetraenoic acid (HETE) and leukotriene A₄) have been implicated certain of inflammatory diseases and cancers.

12-lipoxygenase (12-LOX) exists as three isozymes, platelet-type, leukocyte, and epidermal, but leukocyte 12-LOX is found in rat, mouse, pig and cow, but not in humans. (Yamamoto Biochim. Biophys. Acta. 1992, 1128, 117-131; Funk et al. FEBS Lett. 1997, 402, 162-166). Specifically, 12-LOX has been implicated in diverse conditions such as cancer, types I and II diabetes (including diabetic kidney disease and diabetic neuropathy), thromboses (including heparin induced thrombocytopenia or HIT), inflammatory diseases, neuronal damage associated from an acute ischemic stroke, skin diseases, transplantation, Alzheimer's disease. Fc region receptor (FcγRIIa)-mediated platelet activation, PAR4-AP induced platelet aggregation, anti-phospholipid syndrome; sepsis syndrome; thrombocytopenia associated with immunoglobulin gamma Fc region receptor (FcγRIIa), and non-alcoholic steatohepatitis (NASH). Exemplary 12-LOX inhibitors for use in treating the aforementioned diseases or conditions are described in U.S. Pat. Nos. 10,266,488, 10,752,581, and 11,274,077. A 12(S)-lipoxygenase inhibitor, N-(1,3-benzothiazol-2-yl)-4-[(2-hydroxy-3 methoxyphenyl) methylamino]benzenesulfonamide (ML355), and certain analogs thereof, has been shown to prevent thrombosis in vitro and in vivo. See, e.g., Adili et al. Arterioscler Thromb Vase Biol. 2017; 37:1828.

There remains an ongoing need for treatments for lupus, which has not yet been linked to 12-LOX.

### SUMMARY OF THE INVENTION

The invention provides a method for treating or preventing lupus, comprising administering to a mammal thereof a therapeutically or prophylactically effective amount of a selective 12-LOX inhibitor.

In a specific embodiment, selective 12-LOX-inhibiting compounds of Formula (I) are used in the method:

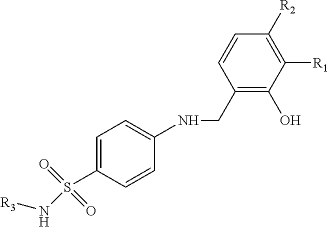

wherein R₁ and R₂ are independently selected from the group consisting of H, alkyl, alkenyl, alkynyl, F, Cl, Br, amine, nitrogen dioxide, indole, alkoxy, cycloalkyl, aryl, heterocycloalkyl, heteroaryl, each optionally substituted with one or more substituents selected from the group consisting of C₁-C₆ alkyl, C₁-C₆ alkenyl, C₁-C₆ alkynyl, F, Cl, Br, hydroxyl, amine, and methoxy;
R₃ is selected from the group consisting of phenyl, cycloalkyl, heterocycloalkyl, aryl, heteroaryl, thiazole, benzothiazole, benzoxazole, imidazole, benzimidazole, thiophene, 1-naphthalene, 2-naphthalene, pyridine, quinoline, isoquinoline, 4N-boc-piperidine-3-phenyl, oxazole, benzothiophene, parathiazine, furan, pyran, chromene, benzofuran, pyrrole, pyrazole, pyrazine, pyrimidine, triazine, indole, purine, phthalazine; each optionally substituted with one or more substituents selected from the group consisting of C₁-C₆ alkyl, C₁-C₆ alkenyl, C₁-C₆ alkynyl, F, Cl, Br, hydroxyl, amine, alkoxy, phenyl, cycloalkyl, aryl, piperazine, piperidine, pyridine, morpholine, pyrrolidine, pyrazolidine, imidazolidine, and thiomorpholine;
or a pharmaceutically acceptable salt thereof, enantiomers thereof, a mixture of enantiomers thereof, or diastereomers thereof.

In a specific embodiment, R₃ is selected from the group consisting of 2-benzothiazole, 2-benzoxazole, 2-benzimidazole, 4-methyl-2-benzothiazole, 2-thiophene, 4-methyl-2-thiazole, 5-methyl-2-thiazole, 5-phenyl-2-thiazole, 4,5-dimethyl-2-thiazole, phenyl, 1-naphthalene, 2-naphthalene, 1,4-bi-phenyl, 3-piperazine-phenyl, 4-piperazine-phenyl, 4-piperidine-phenyl, 4-piperazine-3-pyridine, 6-methyl-3-pyridine, 3-quinoline, 8-isoquinoline, 2-pyridine, 3-pyridine, 3-tertbutyl-phenyl, 6-methoxy-2-benzothiazole, 6-fluoro-2-benzothiazole, 4-phenyl-2-thiazole, 3-morpholine-phenyl, 4N-boc-piperidine-3-phenyl, 3-piperidine-phenyl, 3-isopropyl-phenyl, 3-methoxy-phenyl, 5-methyl-3-isoxazole, 2-pyrimidine and 1,3-bi-phenyl.

In another embodiment, provided is method of treating lupus with a compound of Formula (II):

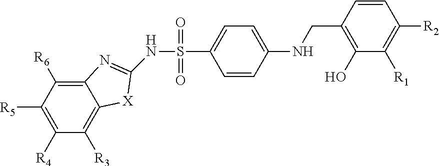

wherein X is selected from the group consisting of O, S, NH, and C;
wherein R₁ and R₂ are independently selected from the group consisting of H, halogen, hydroxyl, alkoxy, and alkyl;
wherein R₃ through R₆ are independently selected from the group consisting of H, halogen, alkoxy, and alkyl;
or a pharmaceutically acceptable salt thereof, enantiomers thereof, a mixture of enantiomers thereof, or diastereomers thereof.

In another aspect, the present invention includes a compound of Formula (III):

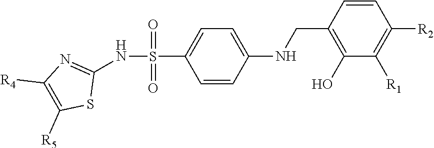

wherein R₁ and R₂ are independently selected from the group consisting of H, halogen, and alkoxy, and further wherein R₁ and R₂ are not both H;
wherein R₄ and R₅ are independently selected from the group consisting of H, alkyl, phenyl, and optionally substituted phenyl;
or a pharmaceutically acceptable salt thereof, enantiomers thereof, a mixture of enantiomers thereof, or diastereomers thereof.

In another aspect, the present invention includes a compound of Formula (IV):

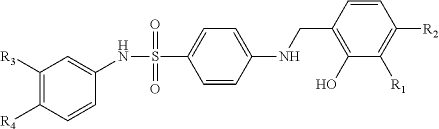

wherein R₁ and R₂ are independently selected from the group consisting of H, halogen, and alkoxy;
wherein R₃ and R₄ are independently selected from the group consisting of H, phenyl, optionally substituted phenyl, tert-butyl, isopropyl, and

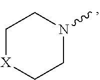

wherein X is selected from the group consisting of NH, O, and

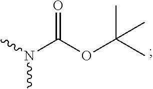

or a pharmaceutically acceptable salt thereof, enantiomers thereof, a mixture of enantiomers thereof, or diastereomers thereof.

In one embodiment, when R₄ is

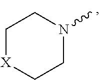

### X is not NH.

In another aspect, the present invention includes a compound of Formula (V):

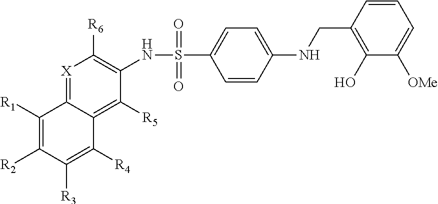

wherein X is selected from the group consisting of N, C, S, and O;
wherein R₁ through R₆ are independently selected from the group consisting of H, alkyl, and alkoxy;
or a pharmaceutically acceptable salt thereof, enantiomers thereof, a mixture of enantiomers thereof, or diastereomers thereof.

In one embodiment, X is C and R₁ through R₆ are H.

In one embodiment, X is N and R₁ through R₆ are H. In another aspect, the present invention includes a compound of Formula (VI):

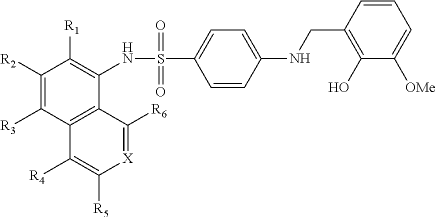

wherein X is selected from the group consisting of N, C, S, and O;
wherein R₁ through R₆ are independently selected from the group consisting of H, alkyl, and alkoxy;
or a pharmaceutically acceptable salt thereof, enantiomers thereof, a mixture of enantiomers thereof, or diastereomers thereof.

In one embodiment, X is C and R₁ through R₆ are H.

In one embodiment, X is N and R₁ through R₆ are H.

In another aspect, the present invention includes a compound of Formula (VII):

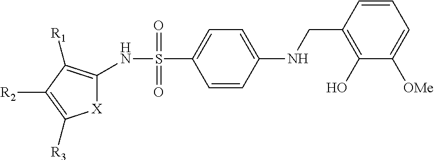

wherein X is selected from the group consisting of N, C, S, and O;
wherein R₁ through R₃ are independently selected from the group consisting of H, alkyl, and alkoxy;
or a pharmaceutically acceptable salt thereof, enantiomers thereof, a mixture of enantiomers thereof, or diastereomers thereof.

In a specific embodiment, the 12-LOX inhibitor is selected from the group consisting of
N-(benzo[d]thiazol-2-yl)-4-[(2-hydroxy-3-methoxybenzyl)amino]benzenesulfonamide;
N-(benzo[d]oxazol-2-yl)-4-((2-hydroxy-3-methoxybenzyl)amino) benzenesulfonamide;
N-(1H-benzo[d]imidazol-2-yl)-4-((2-hydroxy-3-methoxybenzyl)amino) benzenesulfonamide;
4-(2-hydroxy-3-methoxybenzylamino)-N-(thiophen-2-yl)benzenesulfonamide;
4-((2-hydroxy-3-methoxybenzyl)amino)-N-(5-phenylthiazol-2-yl) benzenesulfonamide;
4-((2-hydroxy-3-methoxybenzyl)amino)-N-(3-methoxyphenyl) benzenesulfonamide;
4-(2-hydroxy-3-methoxybenzylamino)-N-(isoquinolin-8-yl) benzenesulfonamide;
4-(2-hydroxy-3-methoxybenzylamino)-N-phenylbenzenesulphonamide;
4-((2-hydroxy-3-methoxybenzyl)amino)-N-(naphthalen-1-yl) benzenesulfonamide;
4-((2-hydroxy-3-methoxybenzyl)amino)-N-(naphthalen-2-yl) benzenesulfonamide;
N-([1,1′-biphenyl]-4-yl)-4-((2-hydroxy-3-methoxybenzyl)amino) benzenesulfonamide;
N-([1,1′-biphenyl]-3-yl)-4-((2-hydroxy-3-methoxybenzyl)amino) benzenesulfonamide;
N-(3-(tert-butyl)phenyl)-4-((2-hydroxy-3-methoxybenzyl)amino) benzenesulfonamide,
4-((2-hydroxy-3-methoxybenzyl)amino)-N-(6-methoxybenzo[d]thiazol-2-yl) benzenesulfonamide,
4-((2-hydroxy-3-methoxybenzyl)amino)-N-(4-phenylthiazol-2-yl) benzenesulfonamide,
tert-butyl4-(3-(4-((2-hydroxy-3-methoxybenzyl) amino) phenylsulfonamido) phenyl) piperidine-1-carboxylate;
4-((2-hydroxy-3-methoxybenzyl)amino)-N-(3-isopropylphenyl) benzenesulfonamide;
N-(6-fluorobenzo[d]thiazol-2-yl)-4-((2-hydroxy-3-methoxybenzyl)amino) benzenesulfonamide;
4-((2-hydroxy-3-methoxybenzyl)amino)-N-(3-(piperazin-1-yl)phenyl) benzenesulfonamide;
4-(2-hydroxy-3-methoxybenzylamino)-N-(4-(piperazin-1-yl)phenyl) benzenesulfonamide; and
4-((2-hydroxy-3-methoxybenzyl)amino)-N-(4-(piperidin-4-yl)phenyl) benzenesulfonamide,
or a pharmaceutically acceptable salt thereof.

In another specific embodiment, the 12-lipoxygenase inhibitor is N-(benzo[d]thiazol-2-yl)-4-[(2-hydroxy-3-methoxybenzyl)amino]benzenesulfonamide.

In one embodiment, the lupus is systemic lupus erythematosus.

In another embodiment, the 12-lipoxygenase inhibitor is formulated for parenteral administration.

In a specific embodiment the 12-lipoxygenase inhibitor is dissolved in a hydroxyalkylated β-cyclodextrin.

In another specific embodiment, the 12-lipoxygenase inhibitor is formulated for oral administration.

In a further embodiment, the invention provides a method of treating lupus with (2S,3S,4S,5R,6S)-6-(2-(((4-(N-(benzo[d]thiazol-2-yl)sulfamoyl)phenyl)amino)methyl)-6-methoxyphenoxy)-3,4,5-trihydroxytetrahydro-2H-pyran-2-carboxylic acid, a metabolite of ML-355 also known as ML-355 glucuroinide, or a pharmaceutically acceptable salt thereof.

### DETAILED DESCRIPTION OF THE INVENTION

It is understood that the present invention is not limited to the particular methodologies, etc. described herein, as these may vary. It is also to be understood that the terminology used herein is used for the purpose of describing particular embodiments only, and is not intended to limit the scope of the present invention.

For therapeutic use. “salts” of the compounds of the present invention will be physiologically acceptable, i.e., the salts will be derived from a physiologically acceptable acid or base. Salts of acids or bases, however, which are not physiologically acceptable may also find use in the preparation or purification of a physiologically acceptable compound. Thus, all salts, whether or not derived form a physiologically acceptable acid or base, are within the scope of the present invention.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

The term “pharmaceutically acceptable carrier,” as used herein, refers to any and all solvents, dispersion media, coatings, antibacterial and antifungal agent, isotonic and absorption delaying agents for pharmaceutical active substances as are well known in the art. Except insofar as any conventional media or agent is incompatible with the compound, its use in the therapeutic compositions is contemplated.

The expression “therapeutically effective amount” refers to an amount of a compound disclosed herein, that is effective for preventing, ameliorating, treating or delaying the onset of a disease or condition and/or that is effective for inhibiting the onset or progression of a disorder. This term also includes mitigating or ameliorating one or more symptoms.

“Alkyl” is C1-C6 hydrocarbon containing normal, secondary, tertiary or cyclic carbon atoms. Examples include, but are not limited to, methyl, ethyl, propyl, isopropyl, tert-butyl, and the like.

“Alkenyl” is C2-C6 hydrocarbon containing normal, secondary, tertiary or cyclic carbon atoms with at least one site of unsaturation, i.e. a carbon-carbon, sp2 double bond. Examples include, but are not limited to, ethylene or vinyl (—CH═CH2), allyl (—CH2CH═CH2), cyclopentenyl (—C5H7), and 5-hexenyl (—CH2CH2CH2CH2CH═CH2), and the like.

“Alkynyl” is C2-C6 hydrocarbon containing normal, secondary, tertiary or cyclic carbon atoms with at least one site of unsaturation, i.e., a carbon-carbon, sp triple bond. Examples include, but are not limited to, acetylenic (—C═—CH) and propargyl (—CH2C═CH), and the like.

“Cycloalkyl” is a saturated, unsaturated or aromatic ring system containing from, for example, 3 to 8 carbon atoms, preferably 3 to 7 carbon atoms, and more preferably 3 to 6 carbon atoms. Examples include, but are not limited to, cyclopentenyl and cyclohexenyl. The cyclic alkyl groups may be unsubstituted or further substituted.

“Heterocycloalkyl” means a saturated, unsaturated or aromatic ring system including at least one N, O, S, or P. Heterocycle thus includes heteroaryl groups. Heterocycle as used herein includes, but is not limited to heterocycles described in PAQUETTE, PRINCIPLES OF MODERN HETEROCYCLIC CHEMISTRY (W. A. Benjamin, New York, 1968), particularly Chapters 1, 3, 4, 6, 7, and 9; THE CHEMISTRY OF HETEROCYCLIC COMPOUNDS, A SERIES OF MONOGRAPHS (John Wiley & Sons. New York, 1950 to present), in particular Volumes 13, 14, 16, 19, and 28; KATRITZKY ET AL., COMPREHENSIVE HETEROCYCLIC CHEMISTRY (Pergamon Press, 1996); and 82 J. AM. CHEM. SOC. 5566 (1960). Heterocycles include, but are not limited to pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, beta-carbolinyl, phenanthridinyl, acridinyl, pyrimidinyl, phenanthrolinyl, phenazinyl, phenothiazinyl, rurazanyl, phenoxazinyl, isochromanyl, chromanyl, imidazolidinyl, imidazolinyl, pyrazolidinyl, pyrazolinyl, piperazinyl, indolinyl, isoindolinyl, quimiclidinyl, morpholinyl, oxazolidinyl, benzotriazolyl, benzisoxazolyl, oxindolyl, benzoxazolinyl, and isatinoyl.

“Aryl” means a monovalent aromatic hydrocarbon radical of 6-20 carbon atoms derived by the removal of one hydrogen atom from a single carbon atom of a parent aromatic ring system. Typical aryl groups include, but are not limited to, radicals derived from benzene, substituted benzene, naphthalene, anthracene, biphenyl, and the like.

“Heteroaryl” means a monovalent aromatic radical of one or more carbon atoms and one or more atoms selected from N, O, S, or P, derived by the removal of one hydrogen atom from a single atom of a parent aromatic ring system. Heteroaryl groups may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S). Heteroaryl bicycles have 7 to 10 ring atoms (6 to 9 carbon atoms and 1 to 2 heteroatoms selected from N, O, and S) arranged as a bicyclo [4,5], [5,5], [5,6], or [6,6] system; or 9 to 10 ring atoms (8 to 9 carbon atoms and 1 to 2 hetero atoms selected from N and S) arranged as a bicyclo [5,6] or [6,6] system. The heteroaryl group may be bonded to the drug scaffold through a carbon, nitrogen, sulfur, phosphorus or other atom by a stable covalent bond. Heteroaryl groups include, but are not limited to, pyridyl, dihydropyridyl isomers, pyridazinyl, pyrimidinyl, pyrazinyl, s-triazinyl, oxazolyl, imidazolyl, thiazolyl, isoxazolyl, pyrazolyl, isothiazolyl, furanyl, thiofuranyl, thienyl, and pyrrolyl.

Unless defined otherwise, all technical and scientific terms used herein have the same meanings as commonly understood by one of ordinary skill in the art to which this invention belongs. Preferred methods and compositions are described, although any methods and compositions similar or equivalent to those described herein can be used in the practice or testing of the present invention.

### 12-Lipoxygenase Inhibitors

In one embodiment, the invention provides a method of treating lupus, comprising administering to a mammal thereof a therapeutically or prophylactically effective amount of any of 12-LOX inhibiting-compound.

A compound or agent is considered a 12-LOX inhibitor if it decreases the function or activity of 12-LOX by at least 10%, at least 20%, at least 30%, at least 40%, at least 50%, at least 60%, at least 70%, at least 80%, at least 90%, or at least 95%. A 12-LOX inhibitor can be an organic compound, an inorganic compound, a biological compound (e.g., proteins or fragments thereof, antibodies or fragments thereof, nucleic acids, nucleic acid analogs, saccharides, or peptides), or any combination thereof. A 12-LOX inhibitor can also be synthetic or naturally occurring.

In one embodiment, the 12-LOX inhibitor is selective for 12-LOX and has little to no activity against other LOX isoforms such as 15-LOX.

Exemplary selective 12-LOX inhibitors are described in U.S. Pat. Nos. 10,266,488, 10,752,581, and 11,274,077, and include ML-355. These compounds are set forth herein. Further included in the methods of the present invention is ML-355 glucuronide, a metabolite of ML-355, whether it is or is not a 12-lipoxygenase inhibitor.

Other non-limiting 12-LOX inhibitors include ETYA (CAS No.: 1191-85-1), baicalein (CAS No.: 491-67-8), 15(S)-HETrE (CAS No.: 13222-49-6), caffeic acid (CAS No.: 331-39-5), CDC (CAS No.: 132465-11-3), esculetin (CAS No.: 305-01-1), ethyl 3,4-dihydroxybenzylidenecyanoacetate (CAS No.: 132464-92-7), ET1 (CAS No.: 13488-22-7), ferulic acid (CAS No.: 1135-24-6), Gossypol (CAS No.: 303-45-7), 2-TEDC (CAS No.: 132465-10-2), Hinokitiol (CAS No.: 499-44-5), 8,11,14-eicosatriynoic acid (CAS No.: 34262-64-1), daphnodorin A, ML355, and a combination thereof. The chemical structure of ML355 is disclosed in Luci et al. (J Med Chem. 2014, 57, 495-506), the content of which is incorporated by reference in its entirety.

12-LOX inhibitors are also disclosed, for example, in US20130096159, WO1990012008, U.S. Pat. Nos. 6,217,875, 4,605,669, 4,623,661, CA1327204. US20070111965, U.S. Pat. No. 4,897,422, US20060193797, U.S. Pat. Nos. 5,120,752, 5,112,848, 5,574,062, EP0416609, U.S. Pat. No. 4,822,811, EP0456760, U.S. Pat. No. 4,761,403, JP2013526576A, the content of each of which is incorporated by reference for its teachings of 12-LOX inhibitors.

In a specific embodiment, the 12-LOX-inhibiting compounds are those of Formula (I)-(VII), described herein, or a salt, enantiomers, a mixture of enantiomers, or diastereomer thereof.

In specific embodiments, exemplary 12-LOX inhibitors for the treatment of lupus are described in U.S. Pat. No. 10,266,488, and include the following compounds:
N-(benzo[d]thiazol-2-yl)-4-[(2-hydroxy-3-methoxybenzyl)amino]benzenesulfonamide;
N-(benzo[d]oxazol-2-yl)-4-((2-hydroxy-3-methoxybenzyl)amino) benzenesulfonamide;
N-(1H-benzo[d]imidazol-2-yl)-4-((2-hydroxy-3-methoxybenzyl)amino) benzenesulfonamide;
4-(2-hydroxy-3-methoxybenzylamino)-N-(thiophen-2-yl)benzenesulfonamide;
4-((2-hydroxy-3-methoxybenzyl)amino)-N-(5-phenylthiazol-2-yl) benzenesulfonamide;
4-((2-hydroxy-3-methoxybenzyl)amino)-N-(3-methoxyphenyl) benzenesulfonamide;
4-(2-hydroxy-3-methoxybenzylamino)-N-(isoquinolin-8-yl) benzenesulfonamide;
4-(2-hydroxy-3-methoxybenzylamino)-N-phenylbenzenesulphonamide;
4-((2-hydroxy-3-methoxybenzyl)amino)-N-(naphthalen-1-yl) benzenesulfonamide;
4-((2-hydroxy-3-methoxybenzyl)amino)-N-(naphthalen-2-yl) benzenesulfonamide;
N-([1,1′-biphenyl]-4-yl)-4-((2-hydroxy-3-methoxybenzyl)amino) benzenesulfonamide;
N-([1,1′-biphenyl]-3-yl)-4-((2-hydroxy-3-methoxybenzyl)amino) benzenesulfonamide;
N-(3-(tert-butyl)phenyl)-4-((2-hydroxy-3-methoxybenzyl)amino) benzenesulfonamide,
4-((2-hydroxy-3-methoxybenzyl)amino)-N-(6-methoxybenzo[d]thiazol-2-yl) benzenesulfonamide,
4-((2-hydroxy-3-methoxybenzyl)amino)-N-(4-phenylthiazol-2-yl) benzenesulfonamide,
tert-butyl4-(3-(4-((2-hydroxy-3-methoxybenzyl) amino) phenylsulfonamido) phenyl) piperidine-1-carboxylate;
4-((2-hydroxy-3-methoxybenzyl)amino)-N-(3-isopropylphenyl) benzenesulfonamide;
N-(6-fluorobenzo[d]thiazol-2-yl)-4-((2-hydroxy-3-methoxybenzyl)amino) benzenesulfonamide;
4-((2-hydroxy-3-methoxybenzyl)amino)-N-(3-(piperazin-1-yl)phenyl) benzenesulfonamide;
4-(2-hydroxy-3-methoxybenzylamino)-N-(4-(piperazin-1-yl)phenyl) benzenesulfonamide; and
4-((2-hydroxy-3-methoxybenzyl)amino)-N-(4-(piperidin-4-yl)phenyl) benzenesulfonamide.
or a pharmaceutically acceptable salt thereof, enantiomers thereof, a mixture of enantiomers thereof, or diastereomers thereof.

In a preferred embodiment, the compound is ML-355, N-(benzo[d]thiazol-2-yl)-4-((2-hydroxy-3-methoxybenzyl)amino) benzenesulfonamide.

In another embodiment, the compound is ML-355 glucuronide.

It is further understood that the above compounds and salts may form solvates, or exist in a substantially uncomplexed form, such as the anhydrous form. When the solvent incorporated in the solvate is water, the molecular complex is a hydrate. Pharmaceutically acceptable solvates include hydrates, alcoholates such as methanolates and ethanolates, acetonitrilates and the like. These compounds can also exist in polymorphic forms.

### Formulation, Administration, and Dosage

The present invention is further directed to treating lupus, or symptoms thereof, comprising administering to a patient in need thereof a at least one compound of Formula (I)-(VII) described herein. Routes of administration and dosages of effective amounts of the pharmaceutical compositions comprising the compounds are also disclosed. The compounds of the present invention can be administered in combination with other pharmaceutical agents in a variety of protocols for effective treatment of disease.

### Formulations

In addition to the compounds disclosed herein, the pharmaceutical compositions of the present invention may further comprise at least one of any suitable auxiliaries including, but not limited to, diluents, binders, stabilizers, buffers, salts, lipophilic solvents, preservatives, adjuvants or the like. Pharmaceutical compositions containing the compounds, salts and hydrates thereof can be prepared by any method known in the art of pharmaceutics. In general, such preparatory methods include the steps of bringing the 12-LOX inhibitor or a pharmaceutically acceptable salt thereof into association with a carrier or excipient, and/or one or more other accessory ingredients, and then, if necessary and/or desirable, shaping, and/or packaging the product into a desired single- or multi-dose unit.

During any of the processes for preparation of the compounds of the present invention, it may be necessary and/or desirable to protect sensitive or reactive groups on any of the molecules concerned. This may be achieved by means of conventional protecting groups, such as those described in PROTECTIVE GROUPS IN ORGANIC CHEMISTRY (1973); and GREENE AND WUTS, PROTECTIVE GROUPS INORGANIC SYNTHESIS (1991). The protecting groups may be removed at a convenient subsequent stage using methods known from the art.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. The composition used in the methods of the present invention may comprise between 0.001% and 100% (w/w) active ingredient, between 0.01% and 100%, 0.01% and 75%, 0.01% and 50%, 0.01% and 25%, 0.01% and 10%, and 0.01 and 5%.

In certain embodiments, the pharmaceutical composition used in the methods of the present invention may comprise a diluent. Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

In certain embodiments, the pharmaceutical composition used in the methods of the present invention may comprise a granulating and/or dispersing agent. Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate, sodium lauryl sulfate, quaternary ammonium compounds, and mixtures thereof.

In certain embodiments, the pharmaceutical composition used in the methods of the present invention may comprise a binding agent. Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate, and larch arabogalactan), alginates, polyethylene oxide, polyethylene glycol, inorganic calcium salts, silicic acid, polymethacrylates, waxes, water, alcohol, and/or mixtures thereof.

In certain embodiments, the pharmaceutical composition used in the methods of the present invention may comprise a preservative. Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

In certain embodiments, the pharmaceutical composition used in the methods of the present invention may comprise an antioxidant. Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

In certain embodiments, the pharmaceutical composition used in the methods of the present invention may comprise a chelating agent. Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

In certain embodiments, the pharmaceutical composition may comprise a buffering agent together with the 12-LOX inhibitor or the salt thereof. Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

In certain embodiments, the pharmaceutical composition used in the methods of the present invention may comprise a lubricating agent. Exemplary lubricating agents include magnesium stearate, calcium stearate, stearic acid, silica, tale, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

### Parenteral Dosage Forms.

In one embodiment, the novel compounds described herein can be administered in a parenteral dosage form. The term “parenteral,” as used herein, includes, but is not limited to, subcutaneous injections, intravenous, intramuscular, intraperitoneal injections, or infusion techniques.

In an embodiment, the parenteral pharmaceutical formulations described herein comprises a compound described herein and a pharmaceutically acceptable carrier.

In one embodiment, the parenteral pharmaceutical formulation may contain liquid carriers, including, vegetable oils such as peanut oil, cotton seed oil, sesame oil, as well as organic solvents, PEG, propylene glycol, glycerol, and surfactants. In certain embodiments for parenteral administration, the conjugates of the invention are mixed with solubilizing agents such as Cremophor™, alcohols, oils, modified oils, glycols, polysorbates, cyclodextrins, polymers, and mixtures thereof.

In another embodiment, the parenteral pharmaceutical formulation is aqueous. In one embodiment, the aqueous parenteral pharmaceutical formulation comprises at least 50% water, preferably 70% or more of water.

In another embodiment, the pharmaceutical carrier comprises a solubilizer. In a specific embodiment, the solubilizer is 2-hydroxyalkylated β-cyclodextrin such as hydroxyethyl β-CD, hydroxypropyl-β-CD, hydroxybutyl β-CD, or sulfobutylether-β-cyclodextrin.

The pharmaceutically acceptable 2-hydroxyalkylated β-cyclodextrin may be present in any suitable amount within the aqueous parenteral pharmaceutical formulations described herein. The pharmaceutically acceptable 2-hydroxyalkylated β-cyclodextrin may be present in amount of between about 5% and 50% w/v. For example, the pharmaceutically acceptable solubilizer, optionally HP-β-CD, may be in an amount of about 10%, 15%, 20%, 25%, 30%, 35%, 40%, 45%, or 50% w/v. In specific embodiments, the HP-β-CD may be present in amount from about 10% to about 40%, from about 20% to about 40%, from about 30% to about 40%, from about 10% to about 30%, or from about 10% to about 20% of the total formulation. In one embodiment, the 2-hydroxyalkylated β-cyclodextrin may present in amount of about 25% of the total formulation w/v.

For parenteral administration, sterile solutions and suspensions are desired. Isotonic preparations which generally contain suitable preservatives are employed when intravenous, administration is desired. The pharmaceutical formulations may be administered parenterally via injection of a pharmaceutical formulation comprising a compound dissolved in an inert liquid carrier, such as sterile water or other pharmaceutically acceptable diluents.

Pharmaceutical compositions or formulations suitable for parenteral administration include aqueous and non-aqueous sterile injection solutions which may contain antioxidants, buffers, bacteriostats and solutes that render the formulation isotonic with the blood of the intended recipient; and aqueous and non-aqueous sterile suspensions which may include suspending agents and thickening agents. The compositions may be presented in unit-dose or multi-dose containers, sealed ampules and vials, and may be stored in a freeze-dried (lyophilized) condition requiring only the addition of the sterile liquid carrier, e.g., water for injections, saline, dextrose in water (D5W), lactated Ringer's solution, immediately prior to use. Extemporaneous immediately prior to use. Extemporaneous injection solutions and suspensions may be prepared from sterile powders, granules and tablets of the kind previously described.

Parenteral formulations may further comprise at least one of any suitable auxiliaries including, but not limited to, diluents, crystal inhibitors, tonicifiers, water structure forming agents or disruptors, polymers, ion pairing agents, stabilizers, buffers, salts, lipophilic solvents, preservatives, adjuvants or the like. Pharmaceutically acceptable auxiliaries are preferred. Examples and methods of preparing such sterile solutions are well known in the art and can be found in well-known texts such as, but not limited to, REMINGTON'S PHARMACEUTICAL SCIENCES (Adejare. Ed., 123rd Edition, Academic Press. (2020); Handbook of Pharmaceutical Excipients, 9^{th} Edition, Pharmaceutical Press (2020)). Pharmaceutically acceptable carriers can be routinely selected that are suitable for the mode of administration, solubility and/or stability of the compound.

Pharmaceutical excipients and additives useful in the parenteral pharmaceutical formulations described herein can also include, but are not limited to, proteins, peptides, amino acids, lipids, and carbohydrates (e.g., sugars, including monosaccharides, di-, tri-, tetra-, and oligosaccharides; derivatized sugars such as alditols, aldonic acids, esterified sugars; and polysaccharides or sugar polymers), which can be present singly or in combination, comprising alone or in combination in ranges of 1-99.99% by weight or volume. Exemplary protein excipients include serum albumin such as human serum albumin (HSA), recombinant human albumin (rHA), gelatin, and casein. Representative amino acid components, which can also function in a buffering capacity, include alanine, glycine, arginine, betaine, histidine, glutamic acid, aspartic acid, cysteine, lysine, leucine, isoleucine, valine, methionine, phenylalanine, and aspartame.

Carbohydrate excipients suitable for use in the parenteral pharmaceutical formulations described herein include but are not limited to monosaccharides such as dextrose, fructose, maltose, galactose, glucose, D-mannose, and sorbose; disaccharides, such as lactose, sucrose, trehalose, and cellobiose; polysaccharides, such as raffinose, melezitose, maltodextrins, dextrans, and starches; and alditols, such as mannitol, xylitol, maltitol, lactitol, xylitol, sorbitol (glucitol), and myoinositol.

The pharmaceutical formulations comprising the compounds described herein can also a pH adjusting agent. The pH adjusting agent may be a base, optionally sodium hydroxide. The pH adjusting agent may also be an acid, optionally hydrochloric acid.

In one embodiment, the present disclosure provides stable parenteral pharmaceutical formulations as well as preserved solutions and formulations containing a preservative, as well as multi-use preserved formulations suitable for pharmaceutical or veterinary use, comprising at least one compound disclosed herein in a pharmaceutically acceptable formulation. Pharmaceutical formulations in accordance with the present disclosure may optionally comprise at least one known preservative. Preservatives include, but are not limited to, phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein. Non-limiting examples include, no preservative, 0.1-2% m-cresol, 0.1-3% benzyl alcohol, 0.001-0.5% thimerosal, 0.001-2.0% phenol, 0.0005-1.0% alkylparaben(s).

Other excipients, e.g., isotonicity agents, buffers, antioxidants, preservative enhancers, can be optionally added. When used, the amount of tonicity modifier used most often ranges from 0.1 to 1% (w/v). Non-limiting examples of suitable tonicity modifiers include sodium chloride, glycerin, boric acid, calcium chloride, dextrose, and potassium chloride. The formulations can include a local anesthetic to reduce the potential of pain during injection. A physiologically tolerated buffer can be added to provide improved pH control if necessary. The pharmaceutical formulations can cover a wide range of pHs, such as from about pH 4 to about pH 10, specifically, a range from about pH 5 to about pH 9, and more specifically, a range of about 7.0 to about 9.0. In one aspect, the formulations described herein have pH between about 8.4 and about 8.7.

The compositions of the present invention can be administered parenterally in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the compounds of the present invention, stabilizers, preservatives, excipients, and the like. The preferred lipids are the phospholipids and the phosphatidyl cholines (lecithins), both natural and synthetic. Methods to form liposomes are known in the art (see Prescott, ed., METH. CELL BIOL. 4:33 (1976)). Liposomes, methods of making and methods of use are described in U.S. Pat. No. 4,089,891 (process for the preparation of liposomes), U.S. Pat. No. 4,233,871 (methods regarding biologically active materials in lipid vesicles), U.S. Pat. No. 4,438,052 (process for producing mixed micelles), U.S. Pat. No. 4,485,054 (large multilamellar vesicles), U.S. Pat. No. 4,532,089 (giant-sized liposomes and methods thereof), U.S. Pat. No. 4,897,269 (liposomal drag delivery system), U.S. Pat. No. 5,820,880 (liposomal formulations), and so forth.

Intravenous doses may include a bolus or a slow dosing. For example, a single bolus can be administered, several divided doses may be administered over time, or the dose may be proportionally reduced or increased as indicated by the exigencies of the therapeutic situation. It can be advantageous to formulate parenteral compositions in dosage unit form for ease of administration and uniformity of dosage.

### Oral Dosage Forms.

The pharmaceutical compositions of the present invention suitable for oral administration may be presented as discrete units such as capsules, dragees, cachets or tablets each containing a predetermined amount of the compound; as a powder or granules; as a solution or a suspension in an aqueous liquid or a non-aqueous liquid; or as an oil-in-water liquid emulsion or a water-in-oil emulsion, and as a bolus, etc.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Suitable pharmaceutically acceptable excipients for oral dosage forms include corn starch, mannitol, povidone, magnesium stearate, talc, cellulose, methylcellulose, carboxymethylcellulose and similar substances. A pharmaceutical composition comprising the compounds and/or a salt thereof may comprise one or more pharmaceutically acceptable excipients, which are known in the art. Formulations include oral films, orally disintegrating tablets, effervescent tablets and granules or beads that can be sprinkled on food or mixed with liquid as a slurry or poured directly into the mouth to be washed down.

For oral administration, the composition also optionally contains a sweetener. Sweeteners include but are not limited to sucrose, fructose, sodium saccharin, sucralose (SPLENDA®), sorbitol, mannitol, aspartame, sodium cyclamate, and the like and combinations thereof.

Oral compositions of the invention include extended- or controlled-release formulations. These may be, for example, diffusion-controlled products, dissolution-controlled products, erosion products, osmotic pump systems or ionic resin systems. Diffusion-controlled products comprise a water-insoluble polymer which controls the flow of water and the subsequent egress of dissolved drug from the dosage from. Dissolution-controlled products control the rate of dissolution of the drug by using a polymer that slowly solubilizes or by microencapsulation of the drug—using varying thicknesses to control release. Erosion products control release of drug by the erosion rate of a carrier matrix. Osmotic pump systems release a drug based on the constant inflow of water across a semi permeable membrane into a reservoir which contains an osmotic agent. Ion exchange resins can be used to bind drugs such that, when ingested, the release of drug is determined by the ionic environment within the gastrointestinal tract.

A tablet may be made by compression or molding, optionally with one or more accessory ingredients. Compressed tablets may be prepared by compressing, in a suitable machine, the compound in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated so as to provide a slow or controlled release of the compound therein.

In other embodiments, the pharmaceutical composition of containing a 12-LOX inhibitor or salt thereof will be administered as a liquid oral dosage form. Liquid dosage forms for oral and parenteral administration include pharmaceutically acceptable emulsions, microemulsions, solutions, suspensions, syrups, and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor™, alcohols, oils, modified oils, glycols, polysorbates, cyclodextrins, polymers, and mixtures thereof.

The aqueous suspensions, emulsions and/or elixirs for oral administration of this invention can be combined with various sweetening agents, flavoring agents, such as, but not limited to orange or lemon flavors, coloring agents, such as dye stuffs, natural coloring agents or pigments, in addition to the diluents such as water, glycerin and various combinations, as described herein.

### Topical and Mucosal Dosage Forms.

Topical and mucosal dosage forms provided herein include, but are not limited to, sprays, aerosols, solutions, emulsions, suspensions, eye drops or other ophthalmic preparations, or other forms known to one of skill in the art. Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed herein are well known to those skilled in the pharmaceutical arts, and depend on the particular tissue to which a given pharmaceutical composition or dosage form will be applied. In one embodiment, excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms.

Methods of preparing the pharmaceutical preparations described herein are manufactured in a manner that is known, including conventional mixing, dissolving, or lyophilizing processes. Thus, aqueous pharmaceutical preparations can be obtained by combining the active compounds with solid excipients, optionally grinding the resulting mixture and processing the mixture of granules, after adding suitable auxiliaries for solid dosage forms, if desired or necessary.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

### Administration.

The pharmaceutical compositions of the inventions can be administered to any animal that can experience the beneficial effects of the compounds of the invention. Such animals include humans and non-humans such as domestic animals (e.g., dogs). In a preferred embodiment, the patient is a human.

The invention further relates to the administration of at least one compound disclosed herein by the following routes, including, but not limited to oral, parenteral, subcutaneous, intramuscular, intravenous, intraarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarenal, intraretinal, intraspinal, intrasynovial, intrathoracic, intrauterine, intravesical, bolus, vaginal, rectal, buccal, sublingual, intranasal, iontophoretic means, or transdermal means.

The pharmaceutical compositions of the present invention are administered to a subject in a manner known in the art. The dosage administered will be dependent upon the age, health, and weight of the recipient, kind of concurrent treatment, if any, frequency of treatment, and the nature of the effect desired.

### Dosage.

In general, the compounds disclosed herein may be used alone or in concert with other therapeutic agents at appropriate dosages defined by routine testing in order to obtain optimal efficacy while minimizing any potential toxicity. Pharmaceutical formulations useful in the present invention can contain a quantity of a compound(s) according to this invention in an amount effective to treat or prevent the condition, disorder or disease of the subject being treated. As a non-limiting example, treatment of humans or animals can be provided as a one-time or periodic dosage of a compound of the present invention 0.0001 to about 50,000 mg per patient, per day. The range may more particularly be from about 0.001 mg/kg to 500 mg/kg of body weight per day, about 0.1 to 500 mg/kg; 0.1-100 mg/kg, about 1.0 to 100 mg/kg, about 1.0-75 mg/kg, about 1.0 to 50 mg/kg, about 1.0-25 mg/kg per day, or about 1.0 to 10 mg/kg for adults (at about 60 kg). Additionally, the dosages may be about 0.5-10 mg/kg per day, about 1.0-5.0 mg/kg per day, 5.0-10 mg/kg per day, or equivalent doses as determine by a practitioner, to achieve a serum concentration that is clinically relevant.

The dosage regimen using the 12-LOX inhibitor may be selected in accordance with a variety of factors including type, species, age, weight, sex, medical condition of the patient; the severity of the condition to be treated; the route of administration; the renal and hepatic function of the patient; and the particular compound employed. A physician or veterinarian of ordinary skill can readily determine and prescribe the effective amount of the drag required to prevent, counter, or arrest the progress of the condition.

Optimal precision in achieving concentrations of drug within the range that yields maximum efficacy with minimal toxicity may require a regimen based on the kinetics of the compound's availability to one or more target sites. Distribution, equilibrium, and elimination of a drug may be considered when determining the optimal concentration for a treatment regimen. The dosages of a compound disclosed herein may be adjusted when combined to achieve desired effects. On the other hand, dosages of these various therapeutic agents may be independently optimized and combined to achieve a synergistic result wherein the pathology is reduced more than it would be if either agent were used alone.

In particular, toxicity and therapeutic efficacy of a compound disclosed herein may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ratio LD₅₀/ED₅₀. Compounds exhibiting large therapeutic indices are preferred except when cytotoxicity of the compound is the activity or therapeutic outcome that is desired. Although compounds that exhibit toxic side effects may be used, a delivery system can target such compounds to the site of affected tissue in order to minimize potential damage to uninfected cells and, thereby, reduce side effects. Generally, the compounds of the present invention may be administered in a manner that maximizes efficacy and minimizes toxicity.

Data obtained from cell culture assays and animal studies may be used in formulating a range of dosages for use in humans. The dosages of such compounds lies preferably within a range of circulating concentrations that include the ED₅₀ with little or no toxicity. The dosage may vary within this range depending upon the dosage form employed and the route of administration utilized. For any compound used in the methods of the present invention, the therapeutically effective dose may be estimated initially from cell culture assays. A dose may be formulated in animal models to achieve a circulating plasma concentration range that includes the IC₅₀ (the concentration of the test compound that achieves a half-maximal inhibition of symptoms) as determined in cell culture. Such information may be used to accurately determine useful doses in humans. Levels in plasma may be measured, by high performance liquid chromatography.

More specifically, the pharmaceutical compositions may be administered in a single daily dose, or the total daily dosage may be administered in divided doses of two, three, or four times daily. Doses maybe administered for one week, one month, or over the course of several months, 3, 6, 9 or 12 months, or intervals known in the art, such as during flare ups, and determined to be clinically relevant. As lupus is a chronic disease, doses of the 12-LOX inhibitors may be continued throughout the life of the patient.

### Combination Therapy

In another embodiment of the present invention, the compounds of the present invention can be co-administered to a patient in need thereof with another active ingredient that has a known side effect of treating lupus, including systemic lupus erythematosus. Such agents include but are not limited to non-steroidal anti-inflammatory drugs (NSAIDs), steroids, antibodies, anti-malarials, and immunosuppressants. Such drugs include traditional NSAIDs, COX-2 inhibitors and salicylates (such as aspirin). Anti-malarials include but are not limited to chloroquinine, hydroxychloroquine chloroquinine phosphate, mefloquine, halofantrine, doxycycline, proguanil, quinidine gluconate, artesunate, atovaquone, primaquine and primaquine phosphate, and quinacrine. Corticosteroids include but are not limited to as prenisone, betamethasone, methylprednisolone acetate, hydrocortisone, and dexamethasone. Immunosuppressants include but are not limited to methotrexate, cyclophosphamide, azathioprine, voclosporin, and mycophenolate mofetil.

Exemplary antibodies include but are not limited to anti-CD6 antibodies, anti-CD20 antibody, anti-CD28 antibodies, anti-IL12 antibodies, anti-IL23 antibodies, anti-BDCA 2 antibodies, anti-CD52 antibodies, anti-CD74 antibodies, rituximab, anifrolumab, and belimumab, or combinations thereof.

The additional therapeutic agent can be solubilized or suspended in a preconcentrate (before dilutions with a diluent), added to the preconcentrate prior to dilution, added to the diluted preconcentrate, or added to a diluent prior to mixing with the preconcentrate. The additional therapeutic agent can also be co-administered as part of an independent dosage form, for therapeutic effect. Optionally, the additional therapeutic agent(s) can be present in a first, solubilized amount, and a second, non-solubilized (suspended) amount Such additional therapeutic agent(s) can be any agent(s) having therapeutic or other value when administered to an animal, particularly to a mammal, such as drags, nutrients, and diagnostic agents

### Therapeutic Effect

The methods of this invention can be used to treat a patient who has one or more manifestations of lupus, including, without limitation, systemic lupus erythematosus, lupus nephritis, cutaneous lupus erythematosus, central nervous system (CNS) lupus, cardiovascular manifestations, pulmonary manifestations, hepatic manifestations, hematological manifestations, gastrointestinal manifestations, musculoskeletal manifestations, neonatal lupus erythematosus, childhood systemic lupus erythematosus, drug-induced lupus erythematosus or complement deficiency syndromes resulting in lupus manifestations.

Exemplary symptoms associated with lupus that can be prevented, mitigated or ameliorated using the 12-LOX inhibitors include, but are not limited to, for example, joint pain and stiffness; muscle aches, pains, or weakness, fever; malaise; cutaneous manifestations (e.g., skin rashes including the “butterfly” rash); unusual weight loss or weight gain; anemia; neurological or neuropsychiatric manifestations (e.g., trouble thinking, memory problems, confusion, depression, headache, seizures, strokes); kidney problems (e.g., nephritis, e.g., glomerulonephritis); chest pain; sun or light sensitivity; hair loss; Raynaud's phenomenon; vascular lesions or other vascular manifestations), as well as biological concomitants of lupus as disclosed herein or as known in the art (e.g., immune complexes, elevated levels of cytokines (e.g., interferons (e.g., Type I interferons, e.g., IFN-α and/or IFN-β); interleukins (e.g., IL-6, IL-8, IL-1, and IL-18) and TNF-α), elevated levels of antibodies associated with lupus (e.g., antinuclear antibodies (e.g., anti-Smith antibodies, anti-double stranded DNA (dsDNA) antibodies, anti-U1 RNP, SS-a (or anti-Ro), SS-b (or anti-La)), antiphospholipid antibodies, anti-ss DNA antibodies, anti-histone antibodies, or anticardiolipin antibodies), and overexpression of IFN inducible genes.

According to some embodiments, treatment of lupus using the disclose 12-LOX-inhibiting compounds results in at least one clinical outcome selected from the group consisting of: an improvement in renal function, a decrease in anti-dsDNA antibody concentration in the serum, a decrease in anti-Cardiolipin antibody concentration in the serum, an increase in serum concentration of complement factor C3 and an increase in serum concentration of complement factor C4. Each possibility represents a separate embodiment of the present invention.

Without wishing to be bound by any theory or mechanism, administration of the composition of the invention to a subject afflicted with lupus may result in a decrease in expression of Toll-Like Receptor 9 and/or inflammatory cytokines, such as, but not limited to, IL-1, IL-6, IL-17 and interferon-gamma, in CD19⁺/CD27⁺ B cells of the patient.

Reduction in flare ups or length of time between flare ups compared to prior to treatment with the 12-LOX inhibitors is also contemplated as a therapeutic outcome.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

The technology described herein is further illustrated by the following examples which in no way should be construed as being further limiting.

1. A method of treating or preventing lupus comprising administering to a patient in need thereof an effective amount of a selective 12-lipoxygenase inhibitor and a pharmaceutically acceptable carrier.

2. The method according to claim 1, wherein the 12-lipoxygenase inhibitor is a compound of Formula (I): 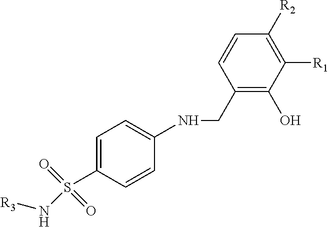
wherein R₁ and R₂ are independently selected from the group consisting of H, alkyl, alkenyl, alkynyl, F, Cl, Br, amine, nitrogen dioxide, indole, alkoxy, cycloalkyl, aryl, heterocycloalkyl, heteroaryl, each optionally substituted with one or more substituents selected from the group consisting of C₁-C₆ alkyl, C₁-C₆ alkenyl, C₁-C₆ alkynyl, F, Cl, Br, hydroxyl, amine, and methoxy;
R₃ is selected from the group consisting of phenyl, cycloalkyl, heterocycloalkyl, aryl, heteroaryl, thiazole, benzothiazole, benzoxazole, imidazole, benzimidazole, thiophene, 1-naphthalene, 2-naphthalene, pyridine, quinoline, isoquinoline, 4N-boc-piperidine-3-phenyl, oxazole, benzothiophene, parathiazine, furan, pyran, chromene, benzofuran, pyrrole, pyrazole, pyrazine, pyrimidine, triazine, indole, purine, phthalazine; each optionally substituted with one or more substituents selected from the group consisting of C₁-C₆ alkyl, C₁-C₆ alkenyl, C₁-C₆ alkynyl, F, Cl, Br, hydroxyl, amine, alkoxy, phenyl, cycloalkyl, aryl, piperazine, piperidine, pyridine, morpholine, pyrrolidine, pyrazolidine, imidazolidine, and thiomorpholine;
or a pharmaceutically acceptable salt thereof, enantiomers thereof, a mixture of enantiomers thereof, or diastereomers thereof.

3. The method according to claim 2, wherein R₃ is selected from the group consisting of 2-benzothiazole, 2-benzoxazole, 2-benzimidazole, 4-methyl-2-benzothiazole, 2-thiophene, 4-methyl-2-thiazole, 5-methyl-2-thiazole, 5-phenyl-2-thiazole, 4,5-dimethyl-2-thiazole, phenyl, 1-naphthalene, 2-naphthalene, 1,4-bi-phenyl, 3-piperazine-phenyl, 4-piperazine-phenyl, 4-piperidine-phenyl, 4-piperazine-3-pyridine, 6-methyl-3-pyridine, 3-quinoline, 8-isoquinoline, 2-pyridine, 3-pyridine, 3-tertbutyl-phenyl, 6-methoxy-2-benzothiazole, 6-fluoro-2-benzothiazole, 4-phenyl-2-thiazole, 3-morpholine-phenyl, 4N-boc-piperidine-3-phenyl, 3-piperidine-phenyl, 3-isopropyl-phenyl, 3-methoxy-phenyl, 5-methyl-3-isoxazole, 2-pyrimidine and 1,3-bi-phenyl.

4. The method according to claim 1, wherein the 12-LOX inhibitor is selected from the group consisting of
N-(benzo[d]thiazol-2-yl)-4-[(2-hydroxy-3-methoxybenzyl)amino]benzenesulfonamide;
N-(benzo[d]oxazol-2-yl)-4-((2-hydroxy-3-methoxybenzyl)amino)benzenesulfonamide;
N-(1H-benzo[d]imidazol-2-yl)-4-((2-hydroxy-3-methoxybenzyl)amino)benzenesulfonamide;
4-(2-hydroxy-3-methoxybenzylamino)-N-(thiophen-2-yl)benzenesulfonamide;
4-((2-hydroxy-3-methoxybenzyl)amino)-N-(5-phenylthiazol-2-yl)benzenesulfonamide;
4-((2-hydroxy-3-methoxybenzyl)amino)-N-(3-methoxyphenyl)benzenesulfonamide;
4-(2-hydroxy-3-methoxybenzylamino)-N-(isoquinolin-8-yl)benzenesulfonamide;
4-(2-hydroxy-3-methoxybenzylamino)-N-phenylbenzenesulfonamide;
4-((2-hydroxy-3-methoxybenzyl)amino)-N-(naphthalen-1-yl)benzenesulfonamide;
4-((2-hydroxy-3-methoxybenzyl)amino)-N-(naphthalen-2-yl)benzenesulfonamide;
N-([1,1′-biphenyl]-4-yl)-4-((2-hydroxy-3-methoxybenzyl)amino)benzenesulfonamide;
N-([1,1′-biphenyl]-3-yl)-4-((2-hydroxy-3-methoxybenzyl)amino)benzenesulfonamide;
N-(3-(tert-butyl)phenyl)-4-((2-hydroxy-3-methoxybenzyl)amino)benzenesulfonamide,
4-((2-hydroxy-3-methoxybenzyl)amino)-N-(6-methoxybenzo[d]thiazol-2-yl)benzenesulfonamide,
4-((2-hydroxy-3-methoxybenzyl)amino)-N-(4-phenylthiazol-2-yl)benzenesulfonamide,
tert-butyl4-(3-(4-((2-hydroxy-3-methoxybenzyl) amino) phenylsulfonamido) phenyl) piperidine-1-carboxylate;
4-((2-hydroxy-3-methoxybenzyl)amino)-N-(3-isopropylphenyl)benzenesulfonamide;
N-(6-fluorobenzo[d]thiazol-2-yl)-4-((2-hydroxy-3-methoxybenzyl)amino) benzenesulfonamide;
4-((2-hydroxy-3-methoxybenzyl)amino)-N-(3-(piperazin-1-yl)phenyl)benzenesulfonamide;
4-(2-hydroxy-3-methoxybenzylamino)-N-(4-(piperazin-1-yl)phenyl)benzenesulfonamide; and
4-((2-hydroxy-3-methoxybenzyl)amino)-N-(4-(piperidin-4-yl)phenyl)benzenesulfonamide,
or a pharmaceutically acceptable salt thereof.

5. The method according to claim 1, wherein the 12-lipoxygenase inhibitor is N-(benzo[d]thiazol-2-yl)-4-[(2-hydroxy-3-methoxybenzyl)amino]benzenesulfonamide.

6. The method according to any of claims 1 to 5, wherein the lupus is systemic lupus erythematosus.

7. The method according to any of claims 1 to 6, wherein the 12-lipoxygenase inhibitor is formulated for parenteral administration.

8. The method according to claim 7, wherein the 12-lipoxygenase inhibitor is dissolved in a hydroxyalkylated β-cyclodextrin.

9. The method according to claim 1, wherein the treatment is amelioration of one or more symptoms of lupus.

10. The method according to claim 9, wherein the symptom is lupus nephritis.

11. A method of treating or preventing lupus comprising administering to a patient in need thereof an effective amount of (2S,3S,4S,5R,6S)-6-(2-(((4-(N-(benzo[d]thiazol-2-yl)sulfamoyl)phenyl)amino)methyl)-6-methoxyphenoxy)-3,4,5-trihydroxytetrahydro-2H-pyran-2-carboxylic acid, or a pharmaceutically acceptable salt thereof.

12. The method according to claim 11, wherein the lupus is systemic lupus erythematosus.

13. The method according to claim 12, wherein the treatment is amelioration of one or more symptoms of systemic lupus erythematosus.

14. The method according to claim 13, wherein the symptom is lupus nephritis.
